(19) United States
(12) Patent Application Publication
(10) Pub. No.: US 2015/0151099 A1
(43) Pub. Date: Jun. 4, 2015

(54) Process of Manufacturing Totally Implantable Venous Device

(71) Applicants: Po-Jen Ko, Taipei City (TW); Ching-Yang Wu, New Taipei City (TW); Yun-Hen Liu, Taoyuan City (TW)

(72) Inventors: Po-Jen Ko, Taipei City (TW); Ching-Yang Wu, New Taipei City (TW); Yun-Hen Liu, Taoyuan City (TW)

(21) Appl. No.: 14/616,789

(22) Filed: Feb. 9, 2015

Related U.S. Application Data
(63) Continuation-in-part of application No. 13/461,889, filed on May 2, 2012.
Application No. 13/461,889 is a continuation-in-part of application No. 12/758,087, filed on Apr. 12, 2010, now abandoned.

(51) Int. Cl. A61M 39/02; B29D 23/00

(52) U.S. Cl. CPC A61M 39/0208; B29D 23/00; A61M 2039/0226; A61M 2205/02; A61M 2207/00; A61M 2210/12; B29K 2705/08

(57) ABSTRACT

A process of manufacturing a totally implantable venous device is provided with forming a catheter; securing a locking nut of the catheter is secured to a proximal end of the catheter; forming a base of injection seat; forming a connecting tube to communicate with injection seat and catheter; putting the locking nut on a bulged central portion of the connecting tube; forming a base and a housing by injection molding with the base of injection seat disposed therein and the locking nut disposed in both the housing and the base; forming a space on a top of the injection seat to communicate with the injection seat; and forming an injection membrane in the space to seal the space.

### CROSS-REFERENCE TO RELATED APPLICATION

This application is a continuation in part of U.S. application Ser. No. 13/461,889, filed May 2, 2012, entitled “TOTALLY IMPLANTABLE VENOUS DEVICE”. The entire teachings of the above application are incorporated herein by reference.

### BACKGROUND OF THE INVENTION

1. Field of Invention

The invention relates to totally implantable venous devices, and particularly relates to a process of manufacturing a totally implantable venous device, the process having advantages including the integral catheter and the port being capable of eliminating space for locking nut and increasing the injection area, the integral implant seat be capable of decreasing the possibility of erroneous assembly, capable of preventing the port from being adversely pressed by the catheter or even broken, and capable of prevent variation of assembly.

2. Description of Related Art

Referring to FIG. 7-8, conventionally, a totally implantable venous device comprises an implant seat 1, a catheter 4, and a locking nut 3. In assembly, the catheter 4 is inserted into the connection portion of the implant seat 1, and, then, the locking nut 3 is put on to fix the catheter 4. Because of the strength difference between the catheter 4 and other components, the catheter 4 fracture may be encountered. And the connection portion of the catheter 4 is susceptible of fracture due to structural weaknesses. When the catheter 4 fracture occurs, the implant seat 1 or the catheter 4 have to be removed because of possible fatal complications. Patients suffered from pain and these lead medical cost increased.

The conventional totally implantable venous devices may be found to have mechanical failure because of more components. In other words, component simplification means improved performance.

Moreover, the implant seat 1 of the conventional totally implantable venous devices is fastened in a subcutaneous pocket of the chest proximate to the armpit. But the body is inclined. In addition, the implant seat 1 of the conventional totally implantable venous devices has a small outer surface 2 which limits the adjustment angle of a non-coring needle 6. And, the non-coring needle 6 is difficult to insert through the outer surface 2, via an injection chamber 5, to the catheter 4.

As shown in FIG. 7, the non-coring needle 6 is perpendicular to the implant seat 1 not to the body curve. However, the inclination of the body can decrease injectable area for non-coring needle 6. This lead difficulties in establishing a secure venous access in obesity patients. Furthermore, the non-coring needle 6 have to be inserted through the thick subcutaneous tissue among these patients and may lead dislodge during patient's arm movement and cause medication extravasation.

In an alternative approach, as shown in FIG. 8, the direction of non-coring needle 6 is perpendicular to tangential direction of the body surface of a patient. All area of injection area could be utilized in this way and dislodge of non-coring needle 6 could be further decreased. However, the dressing of this puncture method is not so convenient because the tail of non-coring needle 6 is not parallel to the body surface and Y-shape gauze needs to be placed beneath the non-coring needle 6 in order to prepare a plan for wound dressing.

Therefore, these disadvantages lead further structural improvement.

### SUMMARY OF THE INVENTION

It is therefore one object of the invention to provide a process of manufacturing a totally implantable venous device comprising the steps of forming a catheter; securing a locking nut over proximal end of the catheter is secured to a connecting site of the catheter; forming an injection seat; forming a connecting tube between injection seat and the catheter; putting the locking nut on a bulged central portion of the connecting tube; forming a base and a housing by injection molding with the injection seat disposed therein and the locking nut disposed in both the housing and the base; forming a space on a top of the injection seat to communicate with the injection seat wherein a chamber is defined by the injection seat; and forming an injection membrane in the space to seal the space.

The above and other objects, features and advantages of the invention will become apparent from the following detailed description taken with the accompanying drawings.

### BRIEF DESCRIPTION OF THE DRAWINGS

FIG. 1 is a flow chart diagram of a process of manufacturing totally implantable venous device according to the invention;

FIG. 2 is an exploded view of a preferred embodiment of a totally implantable venous device according to the invention;

FIG. 3 is a cross-sectional exploded view of the assembled implant seat;

FIG. 4 is a perspective view of the assembled implant seat;

FIG. 5 is a cross-sectional view of the assembled implant seat;

FIG. 6 is an environmental view of the implant seat placed upon the body of a patient;

FIG. 7 is a cross-sectional view of a conventional totally implantable venous device; and

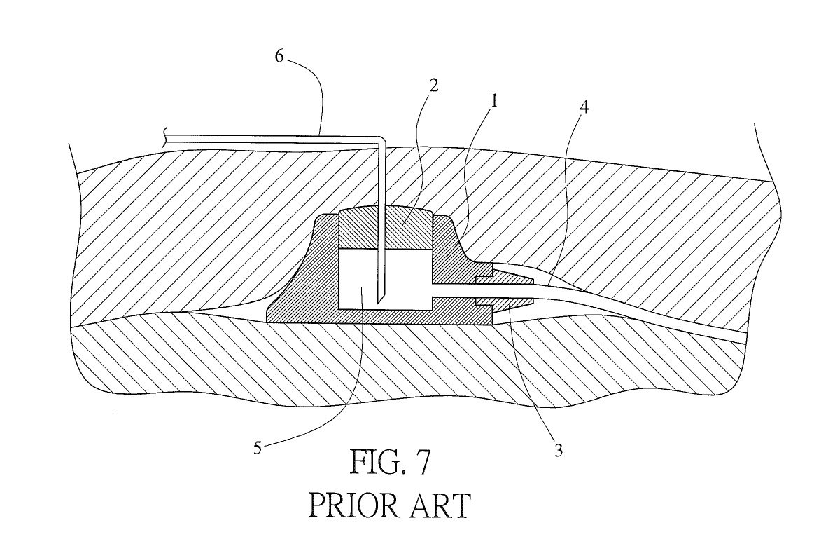

FIG. 8 is another cross-sectional view of a conventional totally implantable venous device.

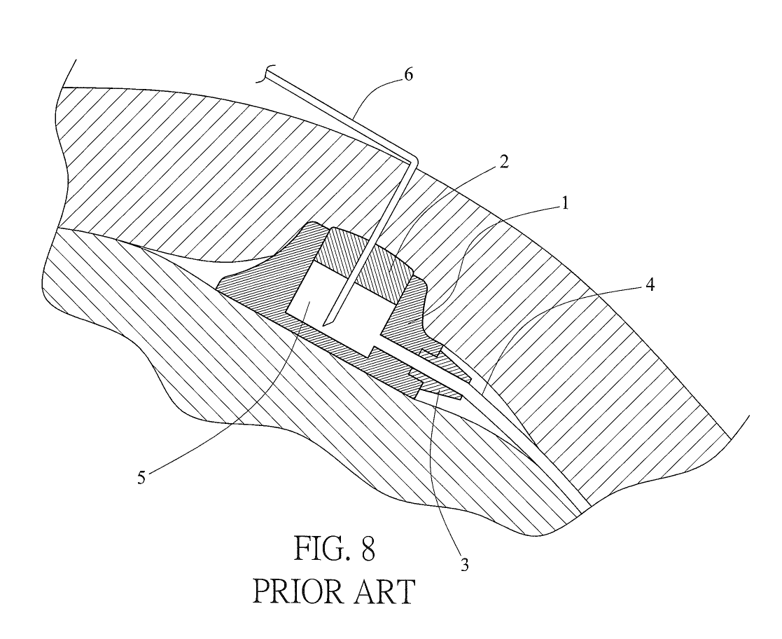

### DETAILED DESCRIPTION OF THE INVENTION

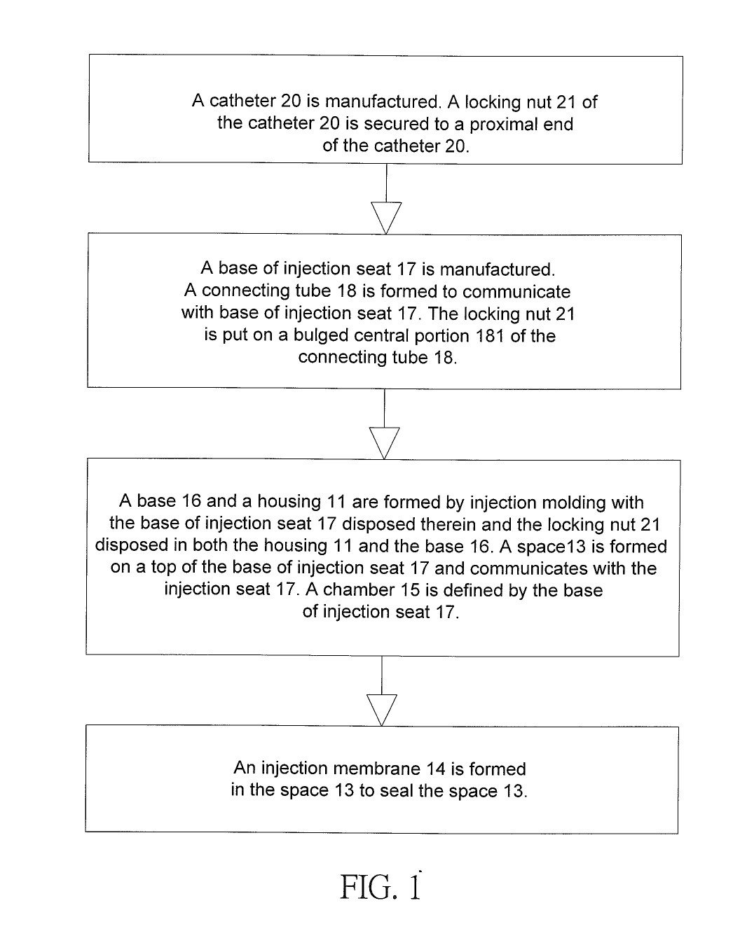

Referring to FIG. 1, a process of manufacturing a totally implantable venous device in accordance with the invention is illustrated. The process comprises the following steps:

First, a catheter 20 is manufactured. Next, a locking nut 21 of the catheter 20 is secured to a proximal end of the catheter 20.

Second, a base of injection seat 17 is manufactured. A connecting tube 18 is formed to communicate with base of injection seat 17. The locking nut 21 is put on a bulged central portion 181 of the connecting tube 18.

Third, a base 16 and a housing 11 are formed by injection molding with the base of injection seat 17 disposed therein and the locking nut 21 disposed in both the housing 11 and the base 16. A space 13 is formed on a top of the base of injection seat 17 and communicates with the injection seat 17. A chamber 15 is defined by the base of injection seat 17.

Fourth, an injection membrane 14 is formed in the space 13 to seal the space 13.

Preferably, tops of both the space 13 and the injection seat 17 are downward inclined an angle from one side of the connector 18 to an opposite side of the connector 18.

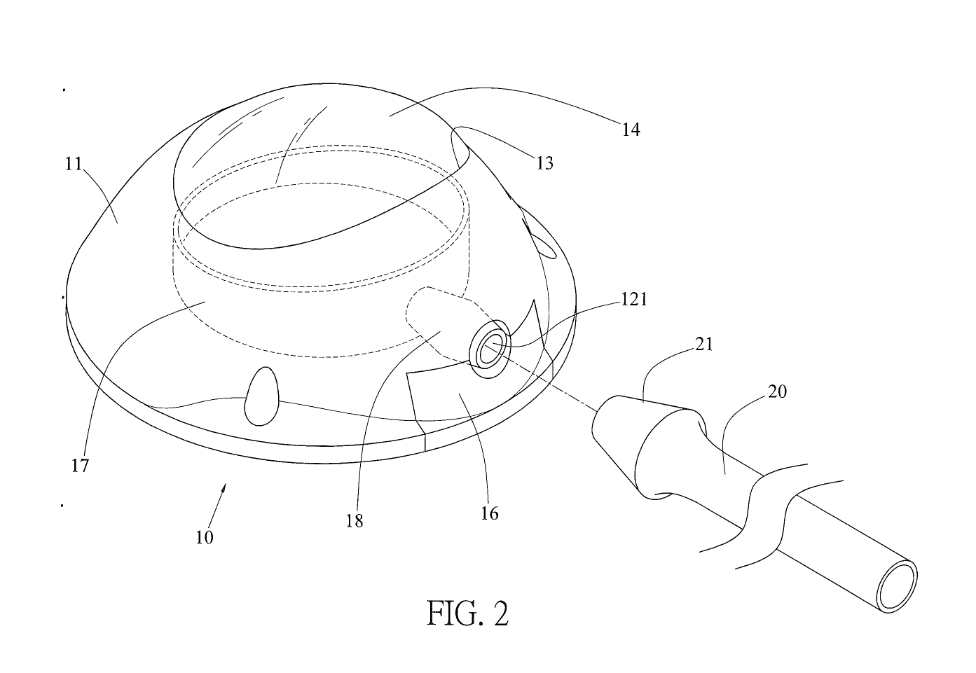

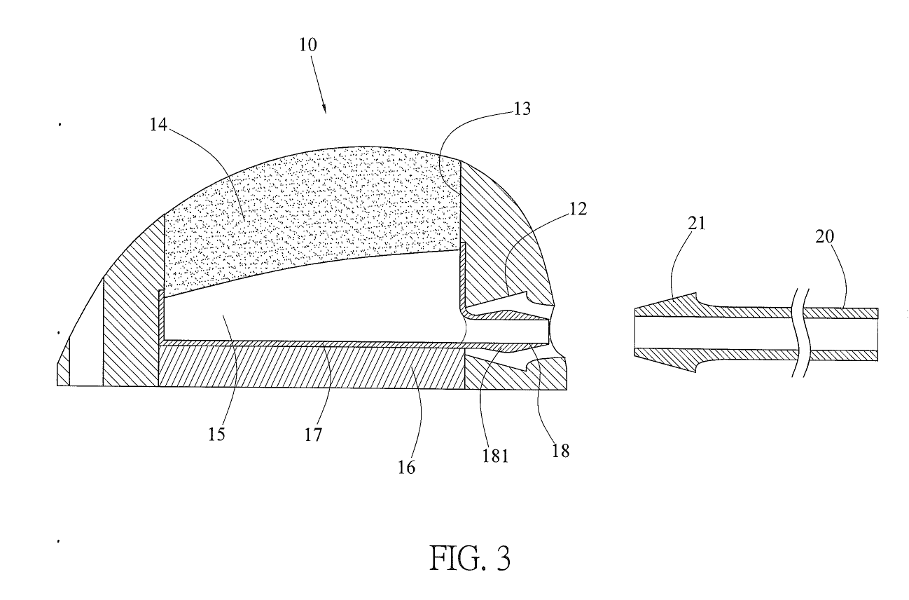

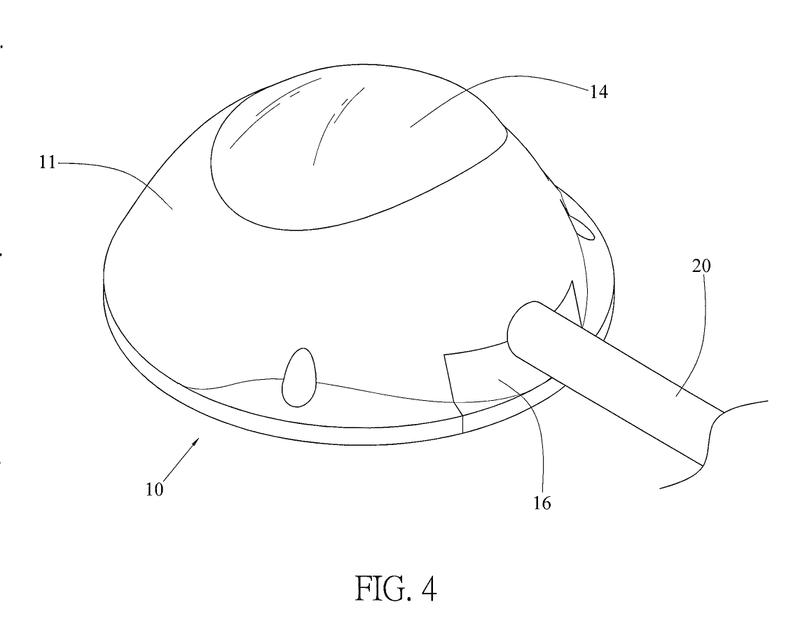

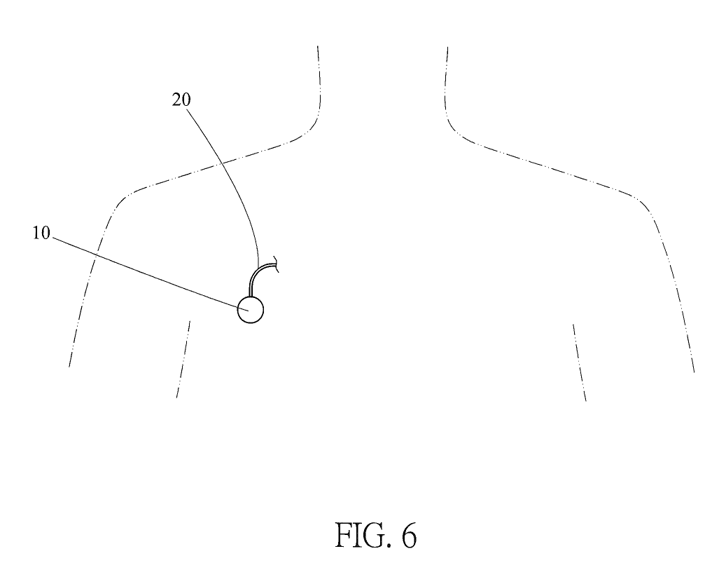

Referring to FIGS. 2 to 6, a totally implantable venous device in accordance with a preferred embodiment of the invention comprises the following components as discussed in detail below.

A hollow port 10 has an ergonomic shape for ease of holding by hand. The port 10 has a housing 11 including a space 13 on a top, an injection membrane 14 sealingly fastened in the space 13 for allowing a needle 60 to be inserted through from one of various angles, an internal chamber 15 under the space 13, and a base 16. The base 16 is structurally strong to prevent the needle 60 from being inserted therethrough.

A peripheral opening 121 having a narrow flat mouth is formed on outer surfaces of both the housing 11 and the base 16. The chamber 15 communicates with the space 13. The space 13 is sealed by the injection membrane 14. A through hole 12 is formed between the chamber 15 and the outer surface of the housing 11 with the opening 121 provided therein.

An injection seat 17 having an open top is fastened in the chamber 15. A connecting tube 18 has one end integrally formed with the base of injection seat 17 and the other end proximate to the mouth of the opening 121. The connecting tube 18 has a bulged central portion 181.

A catheter 20 comprises a locking nut 21 of mushroom shaped at a proximal end. The locking nut 21 forms a watertight structure at the through hole 12 and the opening 121. The locking nut 21 and the connecting tube 18 together form a watertight structure.

Further, the catheter 20 and its locking nut 21 are formed of the same material, and, together, they are not susceptible of breaking. Moreover, the arcuate surface of the housing 11 facilitates holding by the hand in use. The base of injection seat 17 is made of metal resistant to magnetic field (e.g., titanium alloy) or hard plastic. Further, the base of injection seat 17 is resistant to needle insertion and further increases the structural strength of the port 10. The injection membrane 14 is formed of silicone.

As a comparison, conventional totally implantable venous devices have rectangular bodies with four edges in contact with the skin, i.e., frictional contact. This undesirably may cause erosion of overlying skin. This is particularly true to cancer patients. Further, exposed implant seats are required to be removed or re-implanted to a new site. In this invention, the specific arcuate shape leads to less pressure and less friction between the implant seat and overlying skin.

Both totally implantable venous devices of the prior art and the invention are secured onto the chest. The implant seat of the invention has its port 10 being different from that of the prior art. As described above, the port 10 has an arcuate housing 11 which has the advantages of reducing friction with the skin and easy holding by hand.

It is envisaged by the invention that the base of injection seat 17 is fastened in the port 10 with the connecting tube 18 disposed in the through hole 12. The locking nut 21 and catheter 20 is put on the bulged central portion 181 of the connecting tube 18. As a result, the locking nut 21 is fastened in the through hole 12 to form a watertight structure. The base of injection seat 17 is resistant to needle insertion and further increases the structural strength of the port 10 for withstanding pressure exerted by liquid contained in the port 10.

It is noted that the locking nut 21 is mounted in the through hole 12. This has the advantage of preventing the locking nut 21 from being broken due to no protective means as experienced by the locking nut 21 of the conventional port. Further, the bulged central portion 181 can reduce stress generated by the assembled locking nut 21. As a result, the structural strength of the port 10 is greatly increased.

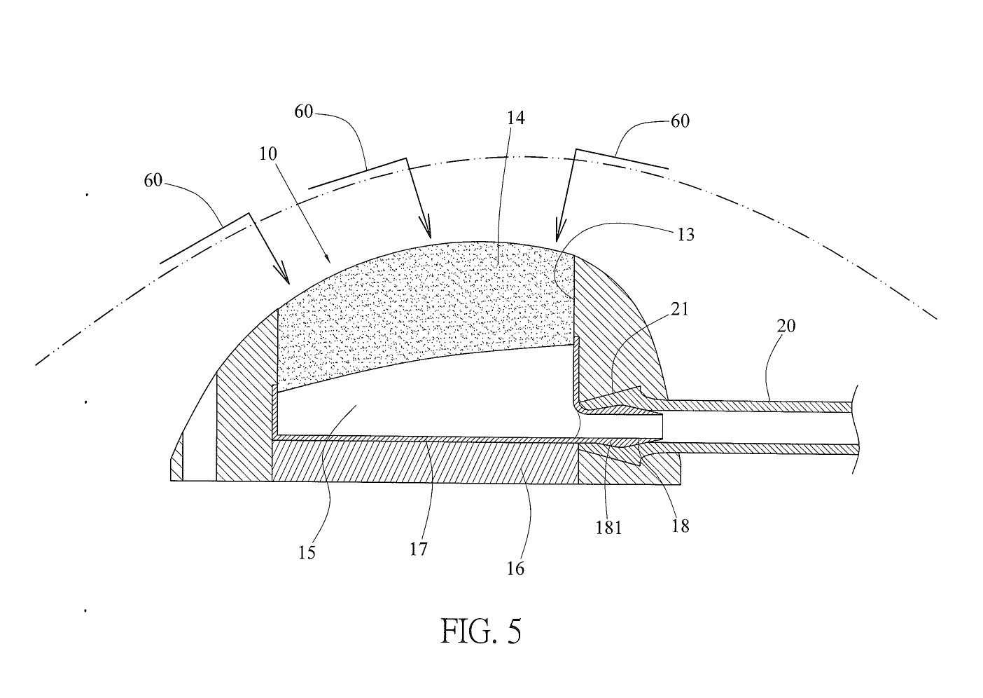

As shown in FIG. 5, the needle 60 can be inserted into the injection membrane 14 from one of various angles. The arcuate housing 11 of the port 10 of the invention can increase the injection area.

On the contrary, the conventional implant seat has a small injection area. The portion connecting the catheter 20 to the port 10 is on an inner surface of the port 10. Hence, it is not easy to bend or even break the catheter 20. On the contrary, the conventional connection point is disposed externally of the port. Hence, it is susceptible of breaking. The catheter 20 and its locking nut 21 are formed integrally. On the contrary, the conventional catheter and its head are separate components. The connecting portion of the port 10 and the catheter 20 are thin to allow vibration of the catheter 20.

The port 10 of the invention is ergonomic in shape. Hence, it is easy to hold the port 10. Further, the injection membrane 14 facilitates non-coring needle insertion. Furthermore, its insertion angle can be adjusted easily. In addition, the arcuate port 10 of the invention has less friction with the skin. The integral catheter 20 and the port 10 can eliminate space for locking nut so that area for injection can be increased. Further, the integral implant seat can decrease the possibility of erroneous assembly. Furthermore, it can prevent the port from being adversely pressed by the catheter or even broken as experienced by the conventional catheter fastening device. In addition, it can prevent variation of assembly as experienced by the prior art.

While the invention herein disclosed has been described by means of specific embodiments, numerous modifications and variations could be made thereto by those skilled in the art without departing from the scope and spirit of the invention set forth in the claims.

What is claimed is:

1. A process of manufacturing a totally implantable venous device comprising the steps of:
forming a catheter;
securing a locking nut of the catheter is secured to a proximal end of the catheter;
forming a base of an injection seat;
forming a connecting tube to communicate with one side of the base of the injection seat;
putting the locking nut on a bulged central portion of the connecting tube;
forming a base and a housing by injection molding with the base of the injection seat disposed therein and the locking nut disposed in both the housing and the base;
forming a space on a top of the injection seat to communicate with the injection seat wherein a chamber is defined by the injection seat; and
forming an injection membrane in the space to seal the space.

2. The process of manufacturing a totally implantable venous device of claim 1, wherein the injection seat is made of metal resistant to magnetic field.

3. The process of manufacturing a totally implantable venous device of claim 2, wherein the metal is a titanium alloy.

4. The process of manufacturing a totally implantable venous device of claim 1, wherein the injection seat is made of hard plastic.

5. The process of manufacturing a totally implantable venous device of claim 1, wherein tops of both the space and the injection seat are downward inclined an angle from one side of the connecting tube to an opposite side of the connecting tube.

6. The process of manufacturing a totally implantable venous device of claim 1, wherein the injection seat is made of silicone.

7. The process of manufacturing a totally implantable venous device of claim 1, wherein the base formed of hard plastic and being capable of preventing a needle from being inserted therethrough.

8. The process of manufacturing a totally implantable venous device of claim 1, wherein the locking nut is mushroom shaped.
